(19) United States
(12) Patent Application Publication
(10) Pub. No.: US 2025/0065345 A1
(43) Pub. Date: Feb. 27, 2025

(54) Nozzle assembly and electric flusher applying same

(71) Applicant: SHENZHEN JINGXINTAI HOUSEWARE CO., LTD., Shenzhen (CN)

(72) Inventors: Jinshan ZOU, Shenzhen (CN); Lin TAN, Shenzhen (CN)

(21) Appl. No.: 18/504,787

(22) Filed: Nov. 8, 2023

(30) Foreign Application Priority Data
Aug. 25, 2023 (CN) 2023110818415

(51) Int. Cl. B05B 1/26; A61M 3/02

(52) U.S. Cl. CPC B05B 1/265; A61M 3/0279; A61M 2206/10

(57) ABSTRACT

A nozzle assembly and an electric flusher applying same, comprising a nozzle body and a water diversion tip, wherein the water diversion tip comprises a installation and flow channel portion and a spherical end portion connected in sequence, the installation and flow channel portion penetrates and is in a main flow channel pipe body of the nozzle body, liquid in the main flow channel pipe body can flow directionally along a side of the installation and flow channel portion, the spherical end portion is supported by the installation and flow channel portion outside a port of the main flow channel pipe body of the nozzle body, the spherical end portion of the water diversion tip and the port of the main channel pipe body of the nozzle body decide a spray separation, and the liquid in the main flow channel pipe body is sprayed out from the spray separation.

### TECHNICAL FIELD

The present invention relates to the technical field of personal care appliances, in particular to a nozzle assembly and an electric flusher applying same.

### BACKGROUND ART

In the past, wastes in facial cavities, such as earwax, nasal dirt and tartar, have been cleaned by cotton swabs or showers sprinkling water, which is very liable to damage the ear canals, nasal cavities and gums, and then cause infection and inflammation.

At the present time, people pay more and more attention to personal cleaning and health care. With the development of technology, electric irrigators such as ear washers, nose washers and tooth washers, which can be driven by small electric pumps to spray water, are gradually popularized.

Some of the electric flushers in the prior art are not equipped with systematic nozzles, and only ends of the water outlet pipes are used as nozzles to spray water; although other electric flushers are equipped with nozzles, the nozzles provide only water passages with diameters corresponding and coaxial to the outlet pipes. The actual purposes of such nozzles are to prevent the hard water outlet pipes from scratching the ear canals, nasal cavities and gums; for the electric flushers in any of the above-mentioned forms, a reasonable flow structure for the water flow to increase the initial velocity and improve the spray distance of the water flow and the flushing intensity of the dirt are still not available. In summary, how to provide a reasonable nozzle flow channel structure for an electric flusher has become an urgent problem to be solved.

### SUMMARY OF THE INVENTION

An object of the present invention is to provide a nozzle assembly and an electric flusher applying same, and provide a reasonable nozzle flow channel structure for the electric flusher.

In order to achieve the above object, the present invention provides the following technical solution: a nozzle assembly comprises a nozzle body and a water diversion tip; wherein the nozzle body comprises a main channel pipe body and a conductor that are arranged from inside to outside in sequence, the main flow channel pipe body is configured for flow of liquid therein in fixed directions, and the conductor is configured for introducing at least a portion of the nozzle body into a human body chamber; the water diversion tip comprises a installation and flow channel portion and a spherical end portion that are connected in sequence; the installation and flow channel portion in part penetrates and is fixed in a main flow channel pipe body of the nozzle body, liquid in the main flow channel pipe body can flow directionally along a side of the installation and flow channel portion, the spherical end portion is supported by the installation and flow channel portion outside a port of the main flow channel pipe body of the nozzle body; and the spherical end portion of the water diversion tip and the port of the main channel pipe body of the nozzle body define a spray separation, and liquid in the main flow channel pipe body is sprayed out from the spray separation.

In the above technical solution, the port of the main flow channel pipe body of the nozzle body comprises a radial opening; and the spherical end portion of the water diversion tip and the radial opening of the main flow channel pipe body of the nozzle body decide the spray separation, so that the spray separation is in an annular shape.

In the above technical solution, a width of the spray separation is L, a maximum diameter of the radial opening is Dmax, and a minimum diameter of the radial opening is Dmin; and L: Dmin=0.2-0.3, and Dmin: Dmax=0.7-0.9.

In the above technical solution, the port of the main flow channel pipe body of the nozzle body is the radial opening; wherein a plurality of spray flow channels are provided on a surface of the spherical end portion of the water diversion tip; and the spherical end portion of the water diversion tip is tightly abutted against the radial opening of the main flow channel pipe body of the nozzle body, and the spray flow channels of the spherical end portion and an inner wall of the radial opening decide the spray separation.

In the above technical solution, the main flow channel pipe body of the nozzle body is provided with a sleeve-fitting installation portion, a first flow channel and a second flow channel in sequence, and an engage rib is formed between the first flow channel and the second flow channel; an outlet pipe of the electric flusher is adapted to be connected into the sleeve-fitting installation portion of the main flow channel pipe body, and the liquid sprayed by the outlet pipe of the electric flusher is suitable for directional flow in the first flow channel and the second flow channel; a first introducing rib and a second introducing rib extending in a radial direction are arranged at intervals in the installation and flow channel portion of the water diversion tip; and the installation and flow channel portion of the water diversion tip penetrates into the main flow channel pipe body of the nozzle body, and the first introducing rib and the second introducing rib clamp the engage rib in the main flow channel pipe body from both sides respectively so as to fix the water diversion tip.

In the above technical solution, a flow channel groove extending in an axial direction is provided in the installation and flow channel portion of the water diversion tip; and the liquid in the first flow channel and the second flow channel of the main flow channel pipe body can directionally flow along the flow channel groove of the water diversion tip.

In the above technical solution, the second flow channel of the main flow channel pipe body is reduced to be platform-shaped relative to the first flow channel.

In the above technical solution, the sleeve-fitting installation portion of the main flow channel pipe body can be in interference fit with the outlet pipe of the electric flusher; and an outlet pipe engage rib for clamping the outlet pipe of the electric flusher is formed in the sleeve-fitting installation portion.

In the above technical solution, a front end portion and a backflow portion are sequentially provided on the conductor of the nozzle body, and the front end portion is configured for being introduced into the human body chamber, and the backflow portion is configured for being tightly abutted against an outer end of the human body chamber; and the backflow portion is concave in the radial direction to form at least one backflow notch for backflow of the liquid in the human body chamber.

In the above technical solution, there are 1-8 backflow notches along a circumferential direction of the backflow portion.

An electric flusher, comprises the nozzle assembly; wherein the electric flusher further comprises an electric flusher body and the water outlet pipe connected to the electric flusher body and used for spraying liquid; and the main flow channel pipe body of the nozzle body is detachably sleeved on the water outlet pipe of the electric flusher.

Compared with the prior art, the beneficial effect of the present invention are as follows: in the nozzle assembly and electric flusher applying same of the present embodiment, the liquid passes through the main flow channel pipe body, and then is sprayed out in a form of an annular water column through the spray separation decided by the spherical end portion of the water diversion tip and the port of the main channel pipe body of the nozzle body. Due to small flow area of the spray separation, the annular water column can obtain a larger initial velocity, thus obtaining a longer spray distance and a larger washing force, which effectively improves the performance of the electric flusher.

### DESCRIPTION OF DRAWINGS

FIG. 1 is one of perspective views of a nozzle assembly in the present invention.

FIG. 2 is one of the exploded views of the nozzle assembly in the present invention.

FIG. 3 is one of the cross-sectional views of the nozzle assembly in the present invention.

FIG. 4 is the second one of the perspective views of the nozzle assembly in the present invention.

FIG. 5 is the second one of the exploded views of the nozzle assembly in the present invention.

FIG. 6 is the second one of the cross-sectional views of the nozzle assembly in the present invention.

FIG. 7 is a view of several forms of the nozzle assembly in the present invention.

FIG. 8 is a perspective view of the electric flusher in the present invention.

In the drawings: 1—nozzle body; 11—conductor; 111—front end portion; 112—backflow portion; 113—backflow notch; 12—main flow channel pipe body; 121—sleeve-fitting installation portion; 122—first flow channel; 123—second flow channel; 124—engage rib; 125—radial opening; 126—outlet pipe engage rib; 2—water diversion tip; 21—installation and flow channel portion; 211—first introducing rib; 212—second introducing rib; 213—flow channel groove; 22—spherical end portion; 10—spray separation; 20—electric flusher body; and 30—outlet pipe.

### DETAILED EMBODIMENTS

Hereinafter, the technical solution in embodiments of the present invention will be clearly and completely described with reference to the accompanying drawings in the embodiments of the present invention. Obviously, the described embodiments are only some of the embodiments of the present invention, rather than all the embodiments. Based on the embodiments of the present invention, all other embodiments obtained by those of ordinary skill in the art without creative efforts fall within the scope of protection of the present invention.

The embodiment provides a nozzle assembly used as a water outlet end of an electric flusher (such as an ear cleaner, a nasal cleaner, a tooth irrigator, etc.). The present embodiment will take an ear cleaner as an example to illustrate the technical solution for the nozzle assembly in detail.

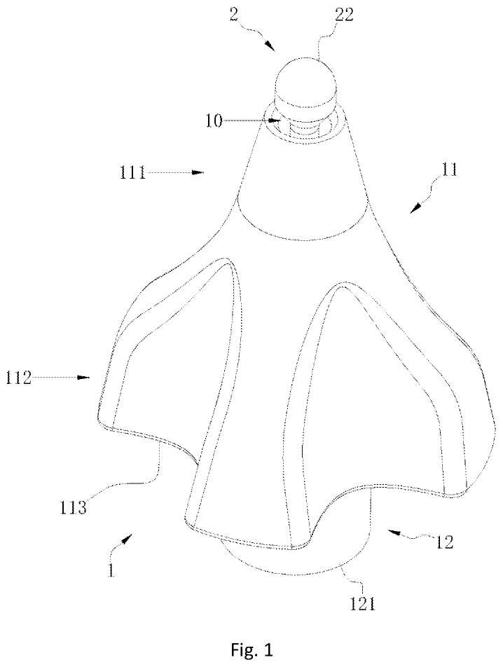

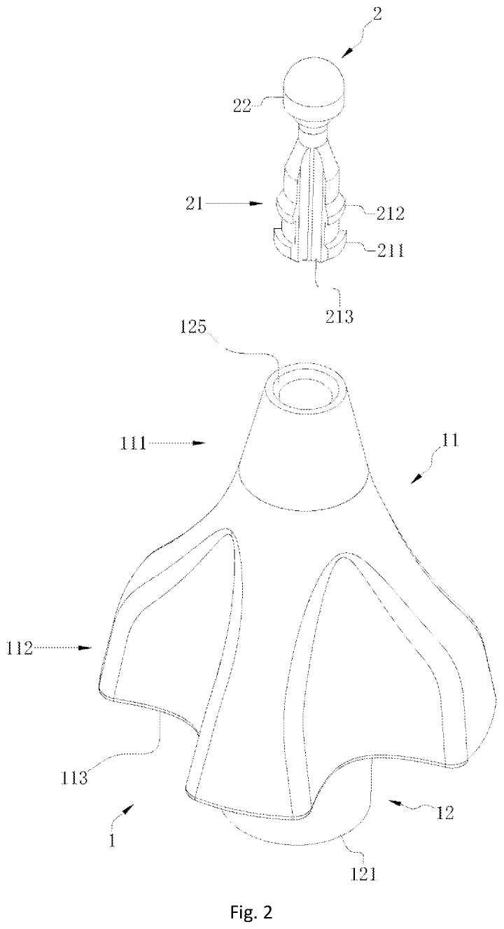

Referring to FIGS. 1-3, the nozzle assembly in the embodiment comprises a nozzle body 1 and a water diversion tip 2.

Wherein, the nozzle body 1 is made of flexible rubber and plastic materials, such as one of medical grade silicone materials, medical grade rubber materials and medical grade soft plastic materials; and the water diversion tip 2 can be made of rubber and plastic or metal materials, such as medical-grade plastic or medical grade stainless steel.

The nozzle body 1 comprises a main channel pipe body 12 and a conductor 11 that are arranged from inside to outside in sequence, the main flow channel pipe body 12 and the conductor 11 are made integrally by injection molding, and both can be connected by ribs arranged in a radial direction, the main flow channel pipe body 12 is suitable for directional flow of liquid (specifically, the liquid sprayed from the outlet pipe 30 of the electric flusher, such as pure water, physiological saline and formulated flushing medicine, etc.) therein, and the conductor 11 is suitable for introducing at least a portion of the nozzle body 1 into a human body chamber (such as the ear canal, nasal cavity and oral cavity, and the embodiment will take the ear canal as an example).

The water diversion tip 2 comprises a installation and flow channel portion 21 and a spherical end portion 22 that are connected in sequence, specifically, the installation and flow channel portion 21 is generally in cylindrical shape, and the spherical end portion 22 is a spherical body with a larger diameter than the installation and flow channel portion 21, and the installation and flow channel portion 21 and the spherical end portion 22 are integrally formed.

The installation and flow channel portion 21 penetrates and is fixed in the main flow channel pipe body 12 of the nozzle body 1, liquid in the main flow channel pipe body 12 can flow directionally along a side of the installation and flow channel portion 21, the spherical end portion 22 is supported by the installation and flow channel portion 21 outside a port of the main flow channel pipe body 12 of the nozzle body, namely, there is a certain distance between the spherical end portion 22 and the port of the main flow channel pipe body 12 of the nozzle body 1.

The spherical end portion 22 of the water diversion tip 2 and the port of the main channel pipe body 12 of the nozzle body 1 define a spray separation 10, and the liquid in the main flow channel pipe body 12 can be sprayed out from the spray separation 10.

Specifically, the main flow channel pipe body 12 of the nozzle body 1 is provided with a sleeve-fitting installation portion 121, a first flow channel 122 and a second flow channel 123 in sequence, and an engage rib 124 is formed between the first flow channel 122 and the second flow channel 123, and in fact, the sleeve-fitting installation portion 121, the first flow channel 122 and the second flow channel 123 are all channel-shaped structures formed inside the main flow channel pipe body 12, the engage rib 124 is an annular rib integrally formed inside the main flow channel pipe body 12; an outlet pipe 30 of the electric flusher is adapted to be connected into the sleeve-fitting installation portion 121 of the main flow channel pipe body 12, and the liquid sprayed by the outlet pipe 12 of the electric flusher is suitable for directional flow in the first flow channel 122 and the second flow channel 123; a first introducing rib 211 and a second introducing rib 212 extending in the radial direction are arranged at intervals in the installation and flow channel portion 21 of the water diversion tip 2; and moreover, at least a surface of the first introducing rib 211 is platform-shaped, and actually, a gap between the first introducing rib 211 and the second introducing rib 212 equals the thickness of the engage rib 124 in the main flow channel pipe body 12; the installation and flow channel portion 21 of the water diversion tip 2 penetrates into the main flow channel pipe body 12 of the nozzle body 1, and the first introducing rib 211 and the second introducing rib 212 clamp the engage rib 124 in the main flow channel pipe body 12 from both sides respectively so as to fix the water diversion tip, and in the above assembly process, the platform-shaped surface of the first introducing rib 211 can introduce the first introducing rib 211 to an inner side of the engage rib 124; and in the above manner, fixed assembly of the nozzle body 1 and the water diversion tip 2 is realized. Further, the second flow channel 123 of the main flow channel pipe body 12 is narrowed to be platform-shaped relative to the first flow channel 122, and due to narrowing of the second flow channel 123, a flow rate of the liquid sprayed from the second flow channel 123 is higher, which improves the washing ability of the liquid.

Further, the sleeve-fitting installation portion 121 of the main flow channel pipe body 12 is in interference fit with the outlet pipe 30 of the electric flusher; and an outlet pipe engage rib 126 for clamping the outlet pipe 30 of the electric flusher is formed in the sleeve-fitting installation portion 121, and in this way, the nozzle body 1 can be quickly assembled and disassembled at a position of the water outlet pipe 30 of the electric flusher.

Further, a flow channel groove 213 extending in an axial direction is provided in the installation and flow channel portion 21 of the water diversion tip 2, and actually, the flow channel groove 213 is a straight groove formed on a surface of the installation and flow channel portion 21; and the liquid in the first flow channel 122 and the second flow channel 123 of the main flow channel pipe body 12 can directionally flow along the flow channel groove 213 of the water diversion tip 2.

Further, the port of the main flow channel pipe body 12 of the nozzle body 1 comprises a radial opening 125, and specifically, a trumpet-shaped notch can be formed at the port of the main flow channel pipe body 12, so as to form the radial opening 125 as an outlet of the second flow channel 123; and the spherical end portion 22 of the water diversion tip 2 and the radial opening 125 of the main flow channel pipe body 12 of the nozzle body 1 decide the spray separation 10, so that the spray separation 10 is in an annular shape.

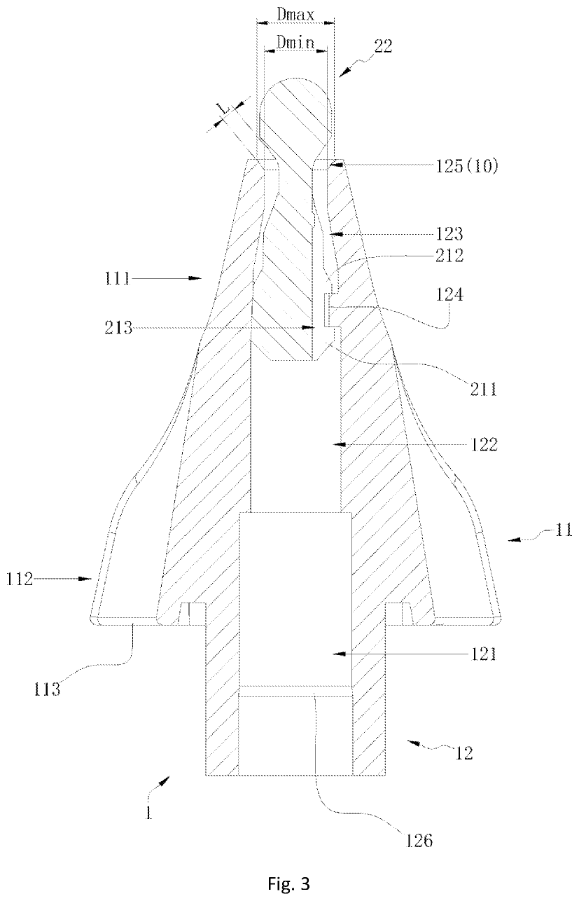

Referring to FIG. 3, further, a width of the spray separation is L, the maximum diameter of the radial opening is Dmax, and the minimum diameter of the radial opening is Dmin; and L: Dmin=0.2˜0.3, and Dmin: Dmax=0.7˜0.9.

Specifically, a front end portion 111 and a backflow portion 112 are sequentially provided on the conductor 11 of the nozzle body 1, a diameter of the front end portion 111 is smaller than that of the backflow portion 112, the front end portion 111 is suitable for being introduced into the human body chamber, and the backflow portion 112 is suitable for being tightly abutted against an outer end of the human body chamber; and the backflow portion 112 is concave in the radial direction to form at least one backflow notch 113 for backflow of the liquid in the human body chamber.

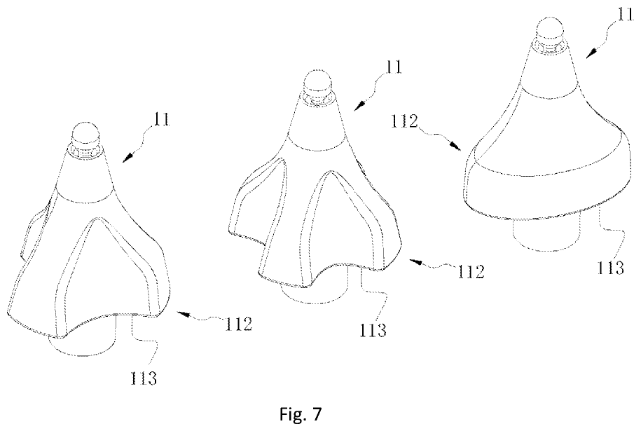

Referring to FIG. 7, further specifically, there are 1-8 backflow notches along a circumferential direction of the backflow portion 113; in some possible embodiments, there are 3 or 4 backflow notches along the circumferential direction of the backflow portion 112, so that the backflow portion 112 is roughly triangular shape or cross-shaped; in other possible embodiments, there are two backflow notches 113 symmetrically on both sides of the backflow portion 112, so that the backflow portion 112 is roughly elliptical.

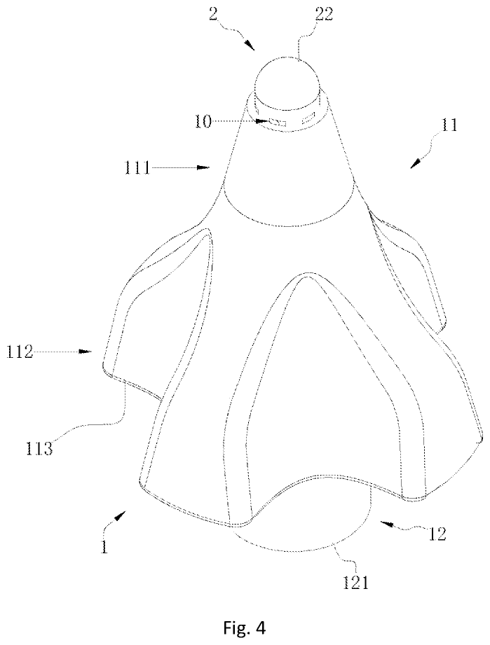

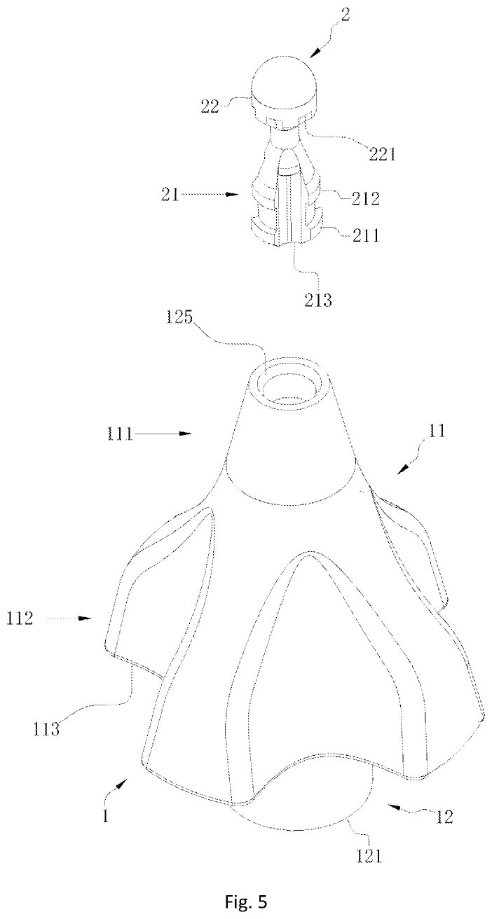

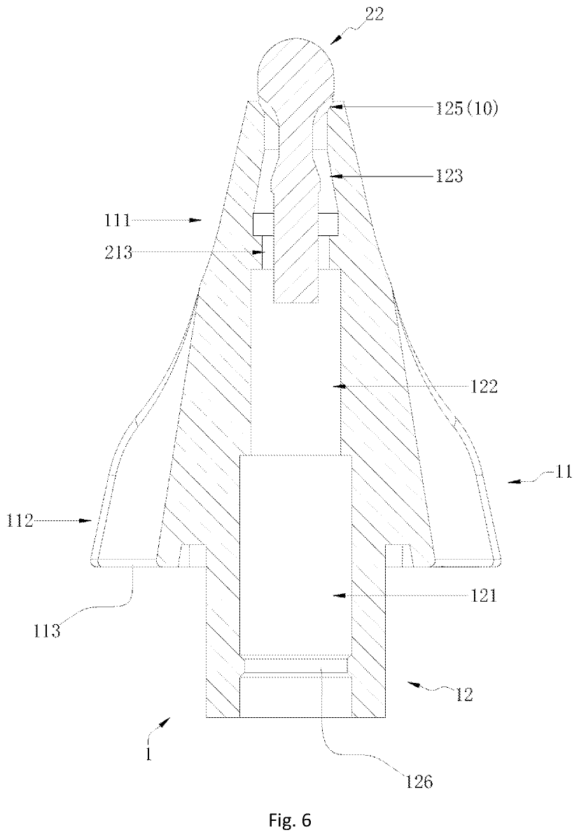

Referring to FIGS. 4-6, in other possible embodiments, the port of the main flow channel pipe body 12 of the nozzle body 1 is a radial opening 125, and specifically, a trumpet-shaped notch can be formed at the port of the main flow channel pipe body 12, so as to form the radial opening 125 as an outlet of the second flow channel 123; a plurality of spray flow channels 221 are provided on a surface of the spherical end portion 22 of the water diversion tip 2; and the spherical end portion 22 of the water distribution tip 2 is tightly abutted against the radial opening 125 of the main flow channel pipe body 12 of the nozzle body 1, and the spray flow channels 221 of the spherical end portion 22 and an inner wall of the radial opening 125 decide the spray separation 10, and at this time, the spray separation 10 is strip-shaped.

The embodiment further provides an electric flusher such as an ear cleaner, a nasal cleaner, a tooth irrigator, etc., and the present embodiment will take an ear cleaner as an example to illustrate.

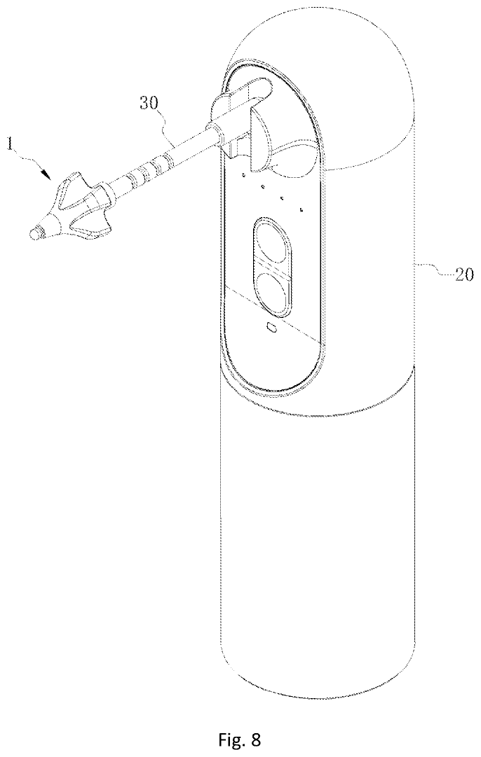

Referring to FIG. 8, the electric flusher in an embodiment comprises the above-mentioned nozzle assembly; wherein it further comprises an electric flusher body 20 and the water outlet pipe 30 connected to the electric flusher body 20 and used for spraying liquid; and the main flow channel pipe body 12 of the nozzle body 1 is detachably sleeved on the water outlet pipe 30 of the electric flusher.

It should be noted that the electric flusher body 20 is internally provided with a liquid storage box, an electric pump for pumping the liquid in the liquid storage box to the water outlet pipe 30 to realize water discharge, and necessary electric control components and connecting pipelines.

It can be understood that the outlet pipe 30 is provided with an annular groove which is suitable for cooperating with the outlet pipe engage rib 126 of the sleeve-fitting installation portion 121 of the main flow channel pipe body 12, which facilitates clamping the nozzle body 1 to on the outlet pipe 30.

When the electric flusher of the embodiment is in use, the electric pump in the electric flusher body 20 is turned on to pump the liquid stored in the liquid storage box to the outlet pipe 30 to realize water discharge, the liquid passes through the first flow channel 122 and the second flow channel 123 of the main flow channel pipe body 12 (flowing along the flow channel groove 213 of the installation and flow channel portion 21) and reaches the radial opening 125, and then is sprayed out in a form of an annular water column through the spray separation 10 decided by the spherical end portion 22 of the water diversion tip 2 and the port of the main channel pipe body 12 of the nozzle body 1. Due to small flow area of the spray separation 10, the annular water column can obtain a larger initial velocity, thus obtaining a longer spray distance and a larger washing force, which effectively improves the performance of the electric flusher.

Although embodiments of the present invention have been shown and described, it will be understood by those skilled in the art that various changes, modifications, substitutions and variations can be made to these embodiments without departing from the principles and spirit of the present invention, the scope of which is defined by the appended claims and equivalents thereof.

1. A nozzle assembly comprises a nozzle body and a water diversion tip;
wherein the nozzle body comprises a main channel pipe body and an conductor arranged from inside to outside in sequence, the main flow channel pipe body is configured for directional flow of liquid therein, and the conductor is configured for introducing at least a portion of the nozzle body into a human body chamber;
the water diversion tip comprises a installation and flow channel portion and a spherical end portion connected in sequence;
the installation and flow channel portion penetrates and is fixed in a main flow channel pipe body of the nozzle body, liquid in the main flow channel pipe body flows directionally along a side of the installation and flow channel portion, the spherical end portion is supported by the installation and flow channel portion outside a port of the main flow channel pipe body of the nozzle body; and
the spherical end portion of the water diversion tip and the port of the main channel pipe body of the nozzle body define a spray separation, and the liquid in the main flow channel pipe body is sprayed out from the spray separation.

2. The nozzle assembly according to claim 1, wherein the port of the main flow channel pipe body of the nozzle body comprises a radial opening; and
the spherical end portion of the water diversion tip and the radial opening of the main flow channel pipe body of the nozzle body decide the spray separation, so that the spray separation is in an annular shape.

3. The nozzle assembly according to claim 1, wherein a width of the spray separation is L, a maximum diameter of the radial opening is Dmax, and a minimum diameter of the radial opening is Dmin; and
then:
L: Dmin=0.2˜0.3, and Dmin: Dmax=0.7˜0.9.

4. The nozzle assembly according to claim 1, wherein the port of the main flow channel pipe body of the nozzle body comprises the radial opening;
a plurality of spray flow channels are provided on a surface of the spherical end portion of the water diversion tip; and
the spherical end portion of the water distribution tip is tightly abutted against the radial opening of the main flow channel pipe body of the nozzle body, and the plurality of spray flow channels of the spherical end portion and an inner wall of the radial opening define the spray separation.

5. The nozzle assembly according to claim 1, wherein the main flow channel pipe body of the nozzle body is provided with a sleeve-fitting installation portion, a first flow channel and a second flow channel in sequence, and an engage rib is formed between the first flow channel and the second flow channel;
an outlet pipe of the electric flusher is adapted to be connected into the sleeve-fitting installation portion of the main flow channel pipe body, and the liquid sprayed by the outlet pipe of the electric flusher is configured to flow directionally in the first flow channel and the second flow channel;
a first introducing rib and a second introducing rib extending in a radial direction are arranged at intervals in the installation and flow channel portion of the water diversion tip; and the installation and flow channel portion of the water diversion tip penetrates into the main flow channel pipe body of the nozzle body, and the first introducing rib and the second introducing rib clamp the engage rib in the main flow channel pipe body from both sides respectively so as to fix the water diversion tip.

6. The nozzle assembly according to claim 5, wherein a flow channel groove extending in an axial direction is provided in the installation and flow channel portion of the water diversion tip; and
the liquid in the first flow channel and the second flow channel of the main flow channel pipe body directionally flows along the flow channel groove of the water diversion tip.

7. The nozzle assembly according to claim 5, wherein the sleeve-fitting installation portion of the main flow channel pipe body is in interference fit with the outlet pipe of the electric flusher; and
an outlet pipe engage rib for clamping the outlet pipe of the electric flusher is formed in the sleeve-fitting installation portion.

8. The nozzle assembly according to claim 1, wherein a front end portion and a backflow portion are sequentially provided on the conductor of the nozzle body, and the front end portion is configured for being introduced into the human body chamber, and the backflow portion is configured for being tightly abutted against an outer end of the human body chamber; and
the backflow portion is concave in the radial direction to form at least one backflow notch for backflow of the liquid in the human body chamber.

9. The nozzle assembly according to claim 8, wherein there are one to eight backflow notches along a circumferential direction of the backflow portion.

10. An electric flusher, comprises the nozzle assembly according to claim 1;
wherein the electric flusher further comprises an electric flusher body and the water outlet pipe connected to the electric flusher body and used for spraying liquid; and
the main flow channel pipe body of the nozzle body is detachably sleeved on the water outlet pipe of the electric flusher.

11. The nozzle assembly according to claim 2, wherein a width of the spray separation is L, a maximum diameter of the radial opening is Dmax, and a minimum diameter of the radial opening is Dmin; and
then:
L: Dmin=0.2˜0.3, and Dmin: Dmax=0.7˜0.9.
